(19) United States
(12) Patent Application Publication
(10) Pub. No.: US 2011/0123591 A1
(43) Pub. Date: May 26, 2011

(54) TUMORICIDAL, BACTERICIDAL, OR VIRICIDAL MACROPHAGE ACTIVATION

(76) Inventors: Charles KNEZEVICH, Spring Valley, CA (US); Robert Silvetz, San Diego, CA (US)

(21) Appl. No.: 12/861,575

(22) Filed: Aug. 23, 2010

Related U.S. Application Data
(60) Provisional application No. 61/236,088, filed on Aug. 22, 2009.

(51) Int. Cl. A61K 9/00; A61K 38/43; A61K 39/395; A61K 38/47; A61P 35/00; A61P 31/04; A61P 31/12; C12N 5/02; C07K 16/00; C12N 11/00

(52) U.S. Cl. 424/422; 424/94.1; 424/178.1; 424/94.3; 435/375; 530/391.7; 435/174

(57) ABSTRACT

The activation of macrophages and methods for treating cancer, bacterial pathogens and viral pathogens are disclosed. In particular, Gc protein is converted to Gc-macrophage activating factor (GcMAF), in vivo or ex vivo. The GcMAF activates macrophages which can then target cancer cells, bacterial pathogens and/or viral pathogens. Alternatively, macrophages are activated by contacting them, in vivo or ex vivo, with GcMAF. Optionally, nagalase is inactivated in a patient receiving the present macrophage activating treatment by contacting the patient's blood with a Nagalase-binding ligand immobilized on an inert medium.

### CROSS-REFERENCE TO A RELATED APPLICATION

This application claims the benefit of U.S. provisional application Ser. No. 61/236,088, filed Aug. 22, 2009, which is incorporated herein by reference in its entirety.

### FIELD OF THE INVENTION

The present invention relates to methods for the activation of macrophages and methods for treating cancer, bacterial pathogens and viral pathogens. In particular, Gc protein is converted to Gc-macrophage activating factor (GcMAF), in vivo or ex vivo. The GcMAF activates macrophages which can then target cancer cells, bacterial pathogens and/or viral pathogens. Alternatively, macrophages are activated by contacting them, in vivo or ex vivo, with GcMAF. Optionally, nagalase is inactivated in a patient receiving the present macrophage activating treatment by contacting the patient's blood with a nagalase binding ligand immobilized on an inert medium.

### BACKGROUND OF THE INVENTION

The uncontrolled growth of metastases resistant to conventional therapeutic modalities is a major cause of death from cancer. Metastases arise from the nonrandom spread of specialized malignant cells that preexist within a primary neoplasm. These metastases can be clonal in their origin, and different metastases can originate from different progenitor cells. In addition, metastatic cells can exhibit an increased rate of spontaneous mutation compared with benign nonmetastatic cells. This data provides an explanation for the clinical observation that multiple metastases can exhibit different sensitivities to the same therapeutic modalities. These findings suggest that the successful therapy of disseminated metastases will have to circumvent the problems of neoplastic heterogeneity and the development of resistance.

Appropriately activated macrophages can fulfill these demanding criteria. Macrophages can be activated to become tumoricidal by interaction with phospholipid vesicles (liposomes) containing immunomodulators. Tumoricidal macrophages can recognize and destroy neoplastic cells in vitro and in vivo, leaving non-neoplastic cells uninjured. Although the exact mechanism(s) by which macrophages discriminate between tumorigenic and normal cells is unknown, it is independent of tumor cell characteristics such as immunogenicity, metastatic potential, and sensitivity to cytotoxic drugs. Moreover, macrophage destruction of tumor cells apparently is not associated with the development of tumor cell resistance.

Additionally, activated macrophages are essential for the immune response to form relative to bacterial and viral invasion. As the mechanism of activation is identical in the three responses (tumoricidal, bactericidal, viricidal), the activation of macrophages has applications across the host immune response, against tumor, bacteria and viral challenges.

Vitamin D-binding protein, also known as DBP or Gc Protein, is an evolutionary conserved glycoprotein among animals (Cooke and Haddad, Endocrine Rev. 10:294 1989). DBP from animals serologically cross-reacts with human DBP (Ogata et al., Comp. Bioch. Physiol. 90B:193, 1988). Animal DBP is a genetically polymorphic plasma protein in some species and has a relative molecular weight of about 52,000. It normally constitutes about 0.5% of the plasma proteins in animals. The plasma concentration is generally about 260 μg/ml. Polymorphism of the human DBP, known as “group specific component” or “Gc protein” is demonstrable by gel electrophoretic analysis, which reveals two major phenotypes: Gc1 and Gc2 (Hirschfeld et al., Nature 185:931, 1960). The entire nucleotide coding sequences of the Gc1 and Gc2 genes, and the predicted amino acid sequences, have been reported (Cooke, et al., J. Clin. Invest. 76:2420, 1985; Yang et al., Proc. Natl. Acad. Sci. USA 82:7994, 1985). Gc1 is further divided into Gc1f and Gc1s subtypes which migrate electrophoretically as two bands, “fast” and “slow”, (Svasti et al., Biochem. 18:1611, 1979).

Activation of macrophages, which is characterized by their consequent enhanced phagocytic activity, is the first major step in a host's immune defense mechanism against cancer and bacterial and viral pathogens. Macrophage activation requires B and T lymphocyte functions, which modify DBP/Gc Protein in a step-wise fashion, to yield GcMAF. Reaction “a” in FIG. 1 shows how Gc protein reacts with beta-galactosidase expressed by B-cells to form an intermediary Gc protein product which then reacts with sialidase expressed by T-cells resulting in the formation of GcMAF

Nagalase (alpha-N-acetylgalactosaminidase), is an enzyme that blocks the conversion of Gc protein to Gc-MAF as shown in reaction (b) in FIG. 1. The nagalase reaction with Gc Protein results in a deglycosylated Gc protein product that prevents the formation of GcMAF and consequent activation of macrophages. Nagalase is produced by many cancer cells and some bacterial and viral pathogens and is a mechanism whereby cancer cells and other pathogens attempt to avoid the host's immune system. Nagalase measurements in blood are used as a diagnostic tool, such as, for example, to diagnose cancer and monitor tumor burden during cancer diagnosis and therapy.

GcMAF should be distinguished from T-cell lymphokine macrophage activating factor, also known as γ-interferon, which is generated by lymphokine-producing T-cells in small amounts, or is obtained by genetic engineering at pharmaceutical grade levels.

Yamamoto U.S. Pat. Nos. 5,177,001; 5,177,002; 5,326,749 and 6,410.269 disclose methods for making GcMAF from Gc protein and smaller domains of the Gc protein resulting in the production of CdMAF. The MAF products made by Yamamoto are then injected into patients to treat cancers and other pathogenic diseases. The present invention provides a novel treatment of activating a patient's own macrophages by in vivo or ex vivo treatments where the macrophages are contacted with MAF or the Gc protein is contacted with enzymes that produce GcMAF. A leukocyte rich fraction of the patient's blood is contacted with the MAF and/or the enzymes which are immobilized on an inert support/medium such as polymer beads or membranes within an aphoretic device or a microfluidic device. Additionally, nagalase can be removed from the patient's blood by immobilizing a nagalase ligand to a solid support and contacting the blood with the immobilized Nagalase ligand.

### SUMMARY OF THE INVENTION

The GcMAF made and used in accordance with the present invention, is created therapeutically in-vivo and/or ex-vivo and from DBP/Gc Protein which is circulating in the plasma of mammalian blood. Additionally, nagalase can be removed from a patient's blood in accordance with the present invention thereby reducing the inhibitory effects of nagalase on the production of GcMAF in the body.

Herein we detail both in-vivo and ex-vivo approaches to the activation of macrophages by means of devices with different strategies of processing the whole blood and plasma all based on standard apheretic and/or microfluidic principles that are well known to one of ordinary skill in the art. In this regard the present invention includes an extracorporeal process where a patient's blood and/or plasma are treated outside of the body and returned to the patient's vascular system Alternatively, a microfluidic device could be implanted in the body to treat macrophages and/or plasma.

Strategy #1: Standard leukapheresis is performed removing at least 500 cc of plasma rich in leukocytes from a mammal. The leukocyte-rich plasma is then passed over a surface containing immobilized GcMAF or Nagalase-binding ligands or both. As a side-effect of direct contact with immobilized GcMAF the macrophages will activate. Any longer-term inhibitory effects from Nagalase on macrophage activation will be mitigated by removal of Nagalase from the plasma by Nagalase-binding ligands. The thus treated leukocyte-rich plasma is then re-transfused back into the mammal to treat a bacterial infection, a viral infection such as hepatitis C or a malignancy.

Strategy #2: A mammal's blood is passed through an apheretic filter containing a fluidized-bed of (a) beads bound with Nagalase-binding ligands, (b) beads bound with beta-galactosidase, (c) beads bound with sialidase or alpha-mannidase, or combinations of (a), (b) and (c). Immune-suppressing Nagalase will be bound in the filter reducing its systemic effect while the beads bound with beta-galactosidase and sialidase, will convert the mammal's own GcProtein into GcMAF for activation of macrophages. The thus treated blood is then re-transfused back into the mammal (patient) to treat a bacterial infection, a viral infection such as hepatitis C or a malignancy.

Strategy #3: In a microfluidic device having a surface with immobilized GcMAF is presented to leukocytes in general, and thus macrophages, activating them. Alternatively, plasma passes over a surface with immobilized beta-galactosidase and immobilized sialidase, to convert the patient's own plasma GcProtein into GcMAF. Additionally, the same surface may also present Nagalase-binding ligands to decrease the effect of that systemic inhibitor.

### BRIEF DESCRIPTION OF THE DRAWINGS

FIG. 1 shows (a) the reaction whereby MAF is made by reactions of GcProtein with enzymes produced by B-cells and T-cells, (b) the deglycosylation reaction of GcProtein by nagalase and (c) the reaction of GcProtein with immobilized enzymes.

FIG. 2 is a flow diagram depicting various treatments made to a mammal's blood by a microfluidic device.

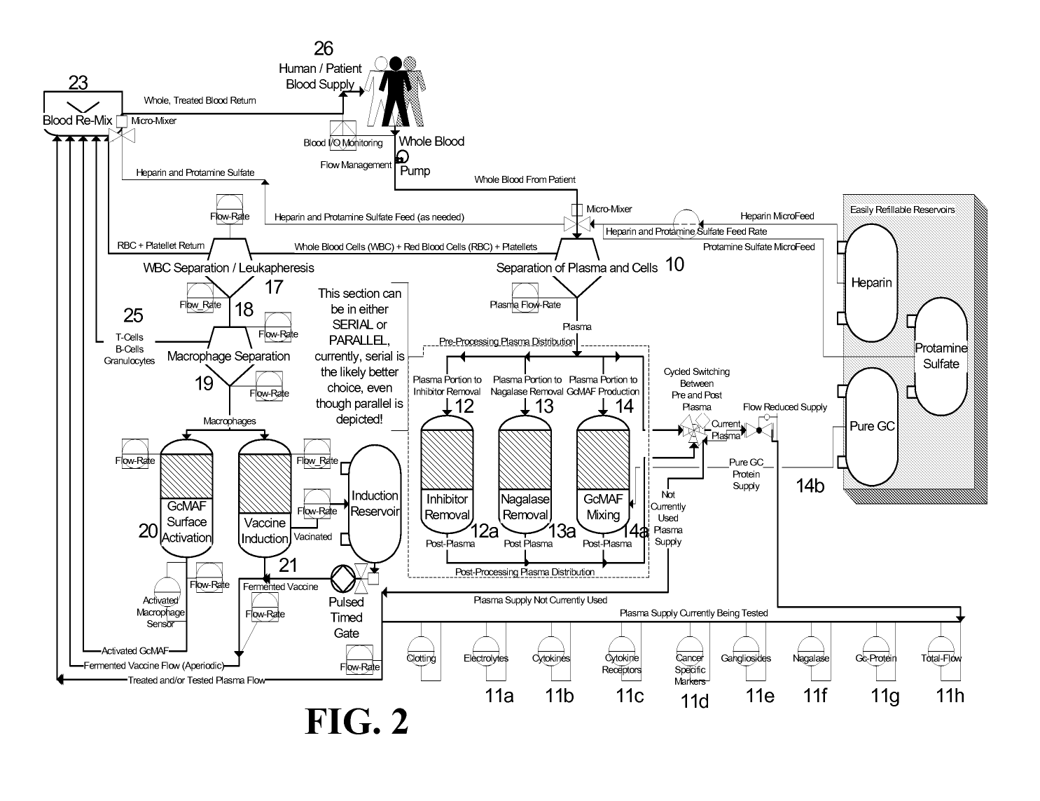

### DETAILED DESCRIPTION OF THE INVENTION

We incorporate herein by reference U.S. Pat. Nos. 5,177,001 and 5,177,002 by Yamamoto, N. concerning the formation of GcMAF from GcProtein in mammals in-vitro. The present invention differentiates itself by performing either direct in-vivo or ex-vivo but real-time exposure of leukocytes to GcMAF or, direct in-vivo or ex-vivo but real-time generation of endogenous GcMAF from circulating GcProtein.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

When used herein the term “extracorporeal device” means any device that is used in a procedure in which blood is taken from a patient's circulation to have a process applied to it before it is returned to the patient's circulation wherein the process can add or subtract either compounds or cells or both in the blood. Suitable extracorporeal devices include, but are not limited to, microfluidic devices, apheresis devices, leukopheresis devices and plasmapheresis devices.

In practicing the present invention, a patient is subjected to in-vivo or ex-vivo treatments where the patient's macrophages are activated and optionally the inhibitory effects of Nagalase are reduced. The patient's macrophages can be activated by (a) contacting a macrophage rich fraction of the patient's blood with GcMAF immobilized to an inert medium or solid support whereby the GcMAF reacts with the macrophages resulting in activation of the macrophages and (b) contacting the patient's plasma that is rich in Gc protein with enzymes that convert Gc protein into GcMAF which can then activate macrophages when the treated plasma is re-introduced back into the patient's vascular system. A Nagalase-binding ligand immobilized to an inert medium can also be employed to reduce the inhibitory effect that nagalase has on the immune system and in particular the deglycosylation of Gc protein.

Microfluidic and microfluidic devices as used herein refer to a laminar flow device that either cell sorts or provides plasma over a biocompatible surface with immobilized binding or catalytic agents. Microfluidic devices may be external to the body with a small pump, or implantable without a pump using pressure drops to drive it. Microfluidic devices are well known and are commercially available from Micronics, Inc., Seattle, Wash., USA. Similarly, leukopheresis and plasmapheresis systems are well known to one of ordinary skill in the art and commercially available from multiple sources.

In one embodiment of the present invention, macrophages are activated in a leukophoretic process where a leukocyte fraction (macrophage rich) of the blood is separated and contacted with immobilized MAF on a biocompatible surface. The activated macrophages in the leukocyte fraction are returned to the patient where the macrophages can perform their immune function in controlling cancers, viral pathogens and bacterial pathogens. The macrophage activation can also be conducted in a microfluidic sorting construct that is intravascular that results in passing macrophages and monocytes over an MAF-immobilized surface at rates of a few ml/minutes, which will result in almost 100% exposure of all known macrophage precursors to the MAF in no more than a 7-day period. The macrophages can then perform their immune function in controlling cancers, viral pathogens and bacterial pathogens.

In another embodiment Nagalase is removed from plasma by subjecting a patient's blood to plasmapheresis and contacting the plasma with immobilized Nagalase-binding ligands where the Nagalase is bound to the ligand and trapped in the electrophoresis apparatus. The treated plasma is returned to the patient minus the bound Nagalase which reduces the immune inhibition that Nagalase normally induces when circulating in the blood. A similar approach is done with a microfluidic device where the plasma is contacted with a Nagalase-binding ligand immobilized within the microfluidic device.

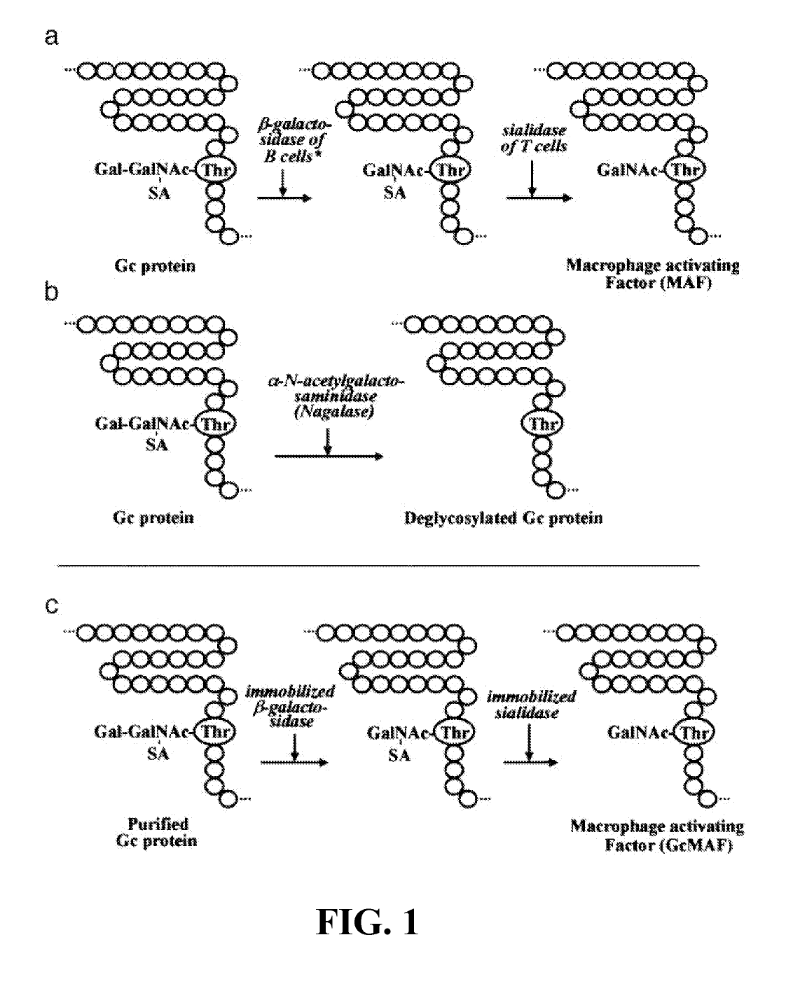

In another embodiment, the GcMAF is made by an apheretic or plasmapheretic process where plasma is separated from whole blood and passed over or contacted with beta-galactosidase and sialidase enzymes that are immobilized on a biocompatible solid support such as beads or a membrane. The beta-galactosidase and sialidase convert the Gc protein into GcMAF. See FIG. 1. The resulting GcMAF produced in the plasma is then re-introduced back into the patient where the GcMAF can activate circulating macrophages. In a preferred embodiment the plasma is also contacted with a Nagalase-binding ligand to decrease the concentration of Nagalase. This lowered concentration of Nagalase allows the GcMAF to interact better in the activation of macrophages when introduced back into the patient's vascular system.

FIG. 1 is a flow diagram showing various treatments made to a mammal's blood by a micro fluidic device. A patient's blood flows into a microfluidic device where a separator 10 separates the plasma from the blood cells (RBC, WBC, platelets). A portion of the plasma is analyzed for diagnostic purposes 11 by measuring the presence/absence of electrolytes 11a, cytokines 11b, cytokine receptors 11c, cancer specific biomarkers 11d, gangliosides 11e, nagalase 11f, GcProtein 11g, total flow 11h or other desired compounds that are indicative of disease or lack thereof. The balance of the plasma, or all of the plasma in the case where no diagnostic tests are run, is then contacted with immobilized ligands to remove specifically targeted compounds from the plasma 12, 13, such as, for example, a Nagalase-binding ligand 12a to remove nagalase and ligands of soluble inhibitors 13a to remove specific soluble inhibitors of the immune system. A list of soluble inhibitors of the immune system 13b include gangliosides; all known growth factors most notably TNF-alpha, TGF-beta and variants, PDGF, EGF, IGF and variants, FGF and variants and VEGF; all known inflammatory cytokine receptors most notably the TNF-alpha family—TNF-R1, TNF-R2, CD40L, NGFR, TRAIL and variants, FASL, IL-1R1, IL1R2, IL-2R, IL-9R, IL-12R, and erythropoietin receptor. The plasma can also be contacted with immobilized enzymes 14 to create new compounds in the plasma such as, for example, the enzymes (beta-galactosidase, sialidase, alpha-mannidase) required to convert GcProtein into Gc-macrophage activating factor (GcMAF) 14a. Optionally, precursor compounds (biological or recombinant) can be added to the plasma in order to increase production of the desired compound. In the case of producing GCMAF, GcProtein is added 14b to the plasma prior to contacting the plasma with the immobilized enzymes 14. After treating the plasma with immobilized enzymes to make compounds or ligands to remove compounds, a portion can be analyzed in a post-plasma treatment diagnostic test 15 to measure the effectiveness of the plasma treatment. Thereafter, the treated plasma is returned to the patient's vascular system with or without combining the plasma with the blood cells separated in 10.

The red blood cells (RBCs), white blood cells (WBCs) and platelets separated in 10 can also be treated. In one embodiment, WBCs are separated from the RBCs and platelets 17 to form a stream having a high concentration of WBCs 18. Further separation of macrophages 19 allows macrophages to be activated by contacting the macrophages with a macrophage activating surface 20 such as immobilized GcMAF or by contacting the macrophages with an antigen to induce a vaccine 21. The treated macrophages can be isolated 22 and administered to the patient or combined back with the patients' plasma 23 and other blood cells for return to the patient's vascular system 2. Alternatively, the vaccine induced macrophages 23 can be optionally retained in a reservoir 24 before being combined back with the WBC fraction that is rich in T cells, B cells and granulocytes 25 and thereafter being returned to the patient's vascular system 26.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of inducing a tumoricidal, bactericidal or viricidal response in a mammal by macrophage activation through the use of an extracorporeal system which comprises contacting a leukocyte fraction of the mammal's blood with (a) GcMAF or (b) one or more enzymes that create endogenous GcMAF from Gc protein precursor.

2. A method of inducing a tumoricidal, bactericidal or viricidal response in a mammal by macrophage activation through the use of an extracorporeal system which comprises reducing the mammal's plasma level of Nagalase by incorporating a Nagalase-binding ligand immobilized on an inert medium in the extracorporeal system.

3. The method of claim 1 in which GcMAF is immobilized on an inert medium and macrophages are exposed to the immobilized GcMAF whereby the macrophages are activated.

4. The method of claim 1 wherein the one or more enzymes are beta-galactosidase, sialidase, alpha-mannosidase or a combination thereof and said enzymes are immobilized on an inert medium.

5. The methods of claim 1 wherein the inert medium can be a hollow fiber, a macroporous bead, a cellulose-based fiber, a synthetic fiber, a silica-based particle, a synthetic membrane, a surface coated with a physiologically-neutral substance, a poly-unsaturated phosphotidylcholine, or a polymer surface.

6. The method of claim 2 wherein a suitable binding ligand is a fragment of a binding partner to which the target binds in nature specifically, a monoclonal antibody, a polyclonal antibody, a designer synthetic peptide, a recombinantly produced monoclonal antibody or a recombinantly produced polyclonal antibody.

7. A method of inducing a tumoricidal, bactericidal or viricidal response in a mammal by macrophage activation through the use of a microfluidic system which comprises implanting in the mammal a microfluidic device that allows the leukocyte fraction to come into contact with GcMAF or one or more enzymes that create endogenous GcMAF from Gc protein precursor.

8. A method of inducing a tumoricidal, bactericidal or viricidal response in a mammal by macrophage activation through the use of a micro fluidic system which comprises implanting in the mammal a microfluidic device that reduces the mammal's plasma level of Nagalase by incorporating a Nagalase-binding ligand immobilized on an inert medium in the microfluidic device.

9. The method of claim 8 wherein a suitable binding ligand is a fragment of a binding partner to which the target binds in nature specifically, a monoclonal antibody, a polyclonal antibody, a designer synthetic peptide, a recombinantly produced monoclonal antibody or a recombinantly produced polyclonal antibody.

10. The method of claim 7 in which GcMAF is immobilized on an inert medium and macrophages are exposed to the immobilized GcMAF.

11. The method of claim 7 wherein the one or more enzymes are beta-galactosidase, sialidase, alpha-mannosidase or a combination thereof and said enzymes are immobilized on an inert medium.

12. The method of claims 7 wherein the inert medium can be a hollow fiber, a macroporous bead, a cellulose-based fiber, a synthetic fiber, a silica-based particle, a synthetic membrane, a surface coated with a physiologically-neutral substance such a poly-unsaturated phosphotidylcholine, or a polymer surface.

13. The method of claim 7 where the microfluidic device is implanted into a mammal's vascular system.

14. The method of claim 7 wherein the microfluidic device is attached to a wearable pump, a wearable plasma separator, a wearable power supply and connected to the vascular system by standard catheters, such that the mammal is fully ambulatory and not tethered to support systems.

15. An extracorporeal device for treating a patient's plasma which comprises a Nagalase-binding ligand immobilized on an inert material whereby Nagalase circulating in the patient's blood binds to the Nagalase-binding ligand.

16. An extracorporeal device for treating a patient's blood which comprises a macrophage activating factor (MAF) immobilized on an inert material whereby macrophages circulating in the patient's blood are activated by the MAF.

17. An extracorporeal device for treating a patient's plasma which comprises enzymes immobilized on an inert material whereby Gc protein circulating in the patient's plasma is converted to GcMAF.

18. The extracorporeal device of claim 16 wherein the enzymes immobilized on the inert material includes (a) a beta-galactosidase and (b) a sialidase, an alpha-mannidase or both a sialidase and an alpha-mannidase.
